(19) United States
(12) Patent Application Publication
(10) Pub. No.: US 2004/0186273 A1
(43) Pub. Date: Sep. 23, 2004

(54) Prion-binding ligands and methods of using same

(75) Inventors: David J. Hammond, Cortland Manor, NY (US); Vite Rose Wiltshire, Plainfield, NY (US); Ruben Carbonell, Raleigh, NC (US); Honglue Shen, Raleigh, NC (US)

Correspondence Address: MINTZ, LEVIN, COHN, FERRIS, GLOVSKY, AND POPEO, P.C., ONE FINANCIAL CENTER, BOSTON, MA, 02111, US

(73) Assignees: V.I. Technologies, Inc., Melville, NY (US); North Carolina State University, Raleigh, NC (US)

(21) Appl. No.: 10/791,516

(22) Filed: Mar. 1, 2004

Related U.S. Application Data
(63) Continuation of application No. 09/543,188, filed on Apr. 5, 2000, now abandoned.

(51) Int. Cl. C07K 7/08; C07K 7/06; C07H 21/04

(52) U.S. Cl. 530/325; 530/326; 530/327; 530/328; 530/329; 536/23.5

(57) ABSTRACT

Disclosed are ligands that bind to regions of the prion peptide and methods of using same.

### FIELD OF THE INVENTION

This invention relates generally to ligands, including peptide ligands, that bind to prion proteins.

### BACKGROUND OF THE INVENTION

Native prion protein, referred to as ‘PrPc’ for cellular prion protein, is widely distributed throughout nature and is particularly well conserved within mammals. The conversion of the native PrPc protein to the infectious protein (referred to as ‘PrPsc’ for scrapie or as ‘PrPres’ for resistant protein) is believed to lead to the propagation of various diseases. Examples of prion associated diseases include, for example, kuru and Creutzfeldt-Jakob disease (CJD) in humans; scrapie in sheep; bovine spongiform encephalopathy (BSE) in cattle; and transmissible mink encephalopathy and wasting disease in deer and elk.

BSE is a form of mad cow disease and is transmissible to a wide variety of other mammals, including humans. The human form of BSE is referred to as new variant Creutzfeldt-Jakob disease, or nvCJD. An estimated 40 million people in the United Kingdom ingested BSE-contaminated beef during the mid- to late 1980s. Because the incubation period for the orally contracted disease may be 20-30 years, the true incidence of this disease may not become apparent until after 2010. The ability to detect and remove prion protein from a sample is of profound importance.

In addition to ingestion of infected beef, there is a potential for transmission of prion associated disease among humans by blood transfusion. The transmissibility of nvCJD is currently unknown. However, it has been found to be present on lymphocytes, and there is evidence indicating that prions are present in the plasma in addition to being cell-associated. Additionally, animals can become infected with prion-associated diseases by grazing on prion-containing soil or by ingesting hay that contains infected hay mites.

### SUMMARY OF THE INVENTION

The invention is based in part on the discovery of ligands that bind to an octapeptide repeat present in the amino acid sequence in prion proteins. Accordingly, in one aspect, the invention includes ligands that bind to polypeptide sequences present in prion proteins. Ligands according to the invention include small molecules, e.g., nucleic acids, nucleic acid analogs, peptides, peptidomimetics, carbohydrates, lipids, small organic and inorganic molecules. Also included in the invention are compounds containing one or more aromatic functionalities such as a porphyrin ring, a phthalocyanine, a napthoquinone, an imidazole, a purine, or a pyrimidine.

The invention also provides compositions containing the prion binding ligands on a support such as a resin or a membrane.

In a further aspect, the invention provides a method of identifying a ligand for a prion protein.

The invention also provides a method for detecting and or removing a prion protein from a sample, e.g., a biological fluid or an environmental sample.

Another aspect of the invention provides a method of treating or retarding the development of a prion-associated pathology in a subject. For example, the ligands of the invention may be useful in treating pathologies such as Creutzfeldt-Jakob disease, Gerstmann-Straussler-Scheinker disease, fatal familial insomnia, scrapie, bovine spongiform encephalopathy, transmissible mink encephalopathy, feline spongiform encephalopathy, exotic ungulate encephalopathy and chronic wasting disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims. In the specification and the appended claims, the singular forms include plural referents unless the context clearly dictates otherwise.

### BRIEF DESCRIPTION OF THE FIGURES

FIG. 1 demonstrates the secondary binding studies of peptide sequences 93, 95, 96 and 98.

FIG. 2 demonstrates the secondary binding studies of PrPc from non-infected prion to acetylated resins containing peptides 84, 85, 96, 98, 101, 111, acetylated control resin (top samples), and non-acetylated peptides: 110, 112, 113, 114, 115, 116 and amino control resin (bottom samples)

FIG. 3 is a representation of Western blot of the binding of prion-from infected brain to various peptide resins

FIG. 4 is a representation of a Western blot showing the binding of prion from infected brain that had been spiked into plasma to acetylated peptide resins.

### DETAILED DESCRIPTION OF THE INVENTION

The present invention provides novel prion-binding ligands useful in methods of detecting and isolating prion protein, as well as methods for diagnosing and treating prion diseases. It also provides methods for screening libraries for ligands to prions, and for removal of prion protein from a sample.

Ligands Binding Prion Polypeptides

Prion binding ligands of the invention are preferably small molecules. “Small molecule” as used herein, is meant to refer to a composition which has a molecular weight of less than about 5 kDa, and preferably less than about 4 kDa. Small molecules can be nucleic acids, peptides, polypeptides, peptidomimetics, carbohydrates, lipids or other organic (carbon containing) or inorganic molecules, and they can be monomeric or polymeric. Many pharmaceutical companies have extensive libraries of chemical and/or biological mixtures, often fungal, bacterial, or algal extracts, which can be used as ligands of the invention.

In one aspect, the invention provides small molecule ligands that bind to peptides or polypeptides derived from the prion protein. No particular length is implied by the term “peptide.” In some embodiments, the peptide is less than, e.g., 100, 75, 50, 25, 20, 17, 15, 12, 10, 9, 8, 7, 6, or 5 amino acids in length. For example, a ligand according to the invention can, in some embodiments, bind to a polypeptide that includes the amino acid sequence GWGQPHGG (SEQ ID NO:1), e.g., a polypeptide having the amino acid sequence GWGQPHGGGWGQPHGG (SEQ ID NO:2).

In some embodiments, the ligand is a peptide that includes an amino acid sequence which binds to a polypeptide derived from the octapeptide region of a prion protein. In various embodiments, the prion-binding peptide includes the amino acid sequence of one or more of SEQ ID NOs:3-30, which are listed in Table 1.

Table 1 shows the peptide sequences determined to bind to the octapeptide through screening. These sequences were synthesized on Toyopearl resin and were tested for their ability to bind to radiolabelled octapeptide repeat in saline. Percentage of binding (%) is the amount of radioactivity that did not bind to the resin using 15 μM starting peptide. Sequences 110 through 119 were screened with plasma containing an additional 100 μM CuCl₂, to ensure saturation of the copper binding sites. The consensus column indicates the presence of aromatic “O” and non-aromatic “x” amino acids.

TABLE 1

% un-
bound

| | | | | | | |
|---|---|---|---|---|---|---|
| Sequence | SEQ ID NO: | Sequence | Library | [¹⁴C] | Consensus | Comments |

| | | | | | | |
|---|---|---|---|---|---|---|
| Control | | | | 80,82 | | Control |
| acetylated | | | | | | |
| Control | | | | 91,93 | | Control |
| amino | | | | | | |
| 84 | SEQ ID NO:3 | LLIWIP | High | 89 | xxxOxx | |
| 85 | SEQ ID NO:4 | WLYWIP | High | 90 | OxOOxx | |
| 86 | SEQ ID NO:5 | WEFYWF | High | 76 | OxOOOO | |
| 87 | SEQ ID NO:6 | YVFNWY | High | 45 | OxOxOO | |
| 88 | SEQ ID NO:7 | LAWFWR | High | 90 | xxOOOx | |
| 89 | SEQ ID NO:8 | GFFFWW | Low | 23 | xOOOOO | |
| 90/98¹ | SEQ ID NO:9 | FYVFTA | Low | 82 | OOxOxx | |
| 91 | SEQ ID NO:10 | YFIWWE | Low | 58 | OOxOOx | |
| 92 | SEQ ID NO:11 | SFPYYY | High | 90 | xOxOOO | |
| 93 | SEQ ID NO:12 | LEIRLA | Low | 96 | xxxxxx | |
| 94 | SEQ ID NO:13 | LLLVIA | Low | 83 | xxxxxx | |
| 95 | SEQ ID NO:14 | SLEEYV | Low | 89 | xxxxOx | |
| 96 | SEQ ID NO:15 | LRVIIS | Low | 90 | xxxxxx | |
| 97 | SEQ ID NO:16 | QLGHQW | Low | 85 | xxxxxO | |
| 99 | SEQ ID NO:17 | SNYGPY | High | 79 | xxOxxO | |
| 100 | SEQ ID NO:18 | PFHPG² | High | 88 | xOxxx | |
| 101 | SEQ ID NO:19 | WIPPYN | High | 84 | OxxxOx | |
| 102 | SEQ ID NO:20 | WFPHFF | High | 64 | OOxxOO | |
| 110 | SEQ ID NO:21 | IQIWIF | High | 85 | xxxOxO | Cu |
| 111 | SEQ ID NO:22 | LWWLFV | High | 85 | xOOxOx | Cu |
| 112 | SEQ ID NO:23 | IFFWIK | High | 77 | xOOOxx | Cu |
| 113 | SEQ ID NO:24 | RWIISL | High | 89 | xOxxxx | Cu |
| 114 | SEQ ID NO:25 | QWWFII | High | 87 | xOOOxx | Cu |
| 115 | SEQ ID NO:26 | VFEYIK | High | 87 | xOxOxx | Cu |
| 116 | SEQ ID NO:27 | WLVWIA | High | 85 | OxxOxx | Cu |
| 117 | SEQ ID NO:28 | YWFIYI | High | 83 | OOOxOx | Cu |
| 118 | SEQ ID NO:29 | TGIPII | High | 88 | xxxxxx | Cu |
| 119 | SEQ ID NO:30 | HKEQGA | Low | 91 | xxxxxx | Cu |

¹The peptide sequence FYVFTA (SEQ ID NO:9) was given two different peptide numbers (90 and 98).
²The 6^{th} position could not be determined.

For example, in some embodiments, the peptide ligand includes an amino acid sequence of LLIWIP (SEQ ID NO:3), WLYWIP (SEQ ID NO:4), WLVWIA (SEQ ID NO:27), IQIWIF (SEQ ID NO:21), IFFWIK (SEQ ID NO:23), and LLLVIA (SEQ ID NO:13).

Preferably, the amino acid sequence of the peptide ligand is not present in the amino acid sequence of a streptavidin polypeptide, e.g., a streptavidin polypeptide having the amino acid sequence of the peptides disclosed in WO 00/02575.

In some embodiments, prion peptides, peptide ligands, and individual moieties or analogs and derivatives thereof, can be chemically synthesized. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al., Intl. J. Peptide Protein Res. 30: 705-739 (1987); Kent, Ann. Rev. Biochem. 57:957-989 (1988); and Kaiser, et al., Science 243: 187-198 (1989). In certain embodiments, the peptides can be synthesized, purified and then coupled to a resin used for screening. In other embodiments, the peptides are synthesized directly on a resin and the resin-bound peptides are then purified.

The peptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of peptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., prion or ligand binding, receptor binding, functional potency or duration of action. See, e.g., Doherty et al., J. Med. Chem. 36: 2585-2594 (1993); Kirby et al., J. Med. Chem. 36:3802-3808 (1993); Morita et al., FEBS Lett. 353: 84-88 (1994); Wang, et al., Int. J. Pept. Protein Res. 42: 392-399 (1993); Fauchere and Thiunieau, Adv. Drug Res. 23: 127-159 (1992).

The prion peptide and the peptide ligands of the invention can be polymers of L-amino acids, D-amino acids, or a combination of both. Also included in the invention are ligands in which analogs of the peptide ligands described herein are present in non-peptidyl linkages.

For example, in various embodiments, the peptide ligands are D retro-inverso peptides. The term “retro-inverso isomer” refers to an isomer of a linear peptide in which the direction of the sequence is reversed and the chirality of each amino acid residue is inverted. See, e.g., Jameson et al., Nature, 368: 744-746 (1994); Brady et al., Nature, 368: 692-693 (1994). The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the invention may be made into an D retro-inverso peptide by synthesizing a reverse of the sequence for the corresponding native L-amino acid sequence. To illustrate, if the peptide model is the prion binding ligand peptide 110: IQIWIF (SEQ ID NO:21), formed of L-amino acids, the retro-inverso peptide analog of this peptide (formed of D-amino acids) would have the sequence, FIWIQI.

Introduction of covalent cross-links into a peptide sequence can conformationally and topographically constrain the peptide backbone. This strategy can be used to develop peptide analogs with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus [e.g., with K₃Fe(CN)₆ at pH 8.5] (Samson et al., Endocrinology, 137: 5182-5185 (1996)), or between two amino acid side chains. See, e.g., DeGrado, Adv. Protein Chem., 39: 51-124 (1988). Disulfide bridges are also introduced into linear sequences to reduce their flexibility. See, e.g., Rose et al., Adv. Protein Chem., 37: 1-109 (1985); Mosberg et al., Biochem. Biophys. Res. Commun., 106: 505-512 (1982).

A number of other methods have been used successfully to introduce conformational constraints into peptide sequences in order to improve their potency, receptor selectivity and biological half-life. These include the use of (i) C_{α}-methylamino acids (see, e.g., Rose et al., Adv Protein Chem., 37: 1-109 (1985); Prasad and Balaram, CRC Crit. Rev. Biochem., 16: 307-348 (1984)); (ii) N_{α}-methylamino acids (see, e.g., Aubry et al., Int. J. Pept. Protein Res., 18: 195-202 (1981); Manavalan and Momany, Biopolymers, 19: 1943-1973 (1980)); and (iii) α,β-unsaturated amino acids (see, e.g., Bach and Gierasch, Biopolymers, 25: 5175-S192 (1986); Singh et al., Biopolymers, 26: 819-829 (1987)). These and many other amino acid analogs are commercially available, or can be easily prepared.

Alternatively, the peptides and peptide ligands may be obtained by methods well-known in the art for recombinant peptide expression and purification. A DNA molecule encoding a peptide according to the invention can be generated. The DNA sequence is deduced from the protein sequence based on known codon usage. See, e.g., Old and Primrose, Principles of Gene Manipulation 3^{rd} ed., Blackwell Scientific Publications, 1985; Wada et al., Nucleic Acids Res. 20: 2111-2118(1992). Preferably, the DNA molecule includes additional sequence, e.g., recognition sites for restriction enzymes which facilitate its cloning into a suitable cloning vector, such as a plasmid. The invention provides the nucleic acids comprising the coding regions, non-coding regions, or both, either alone or cloned in a recombinant vector, as well as oligonucleotides and related primer and primer pairs corresponding thereto. Nucleic acids may be DNA, RNA, or a combination thereof. Vectors of the invention may be expression vectors. Nucleic acids encoding peptides according to the invention may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like). Nucleic acids can also be generated by chemical synthesis.

In some embodiments, a peptide ligand according to the invention binds to a prion polypeptide in the presence of a metal. An example of a metal is copper. While not wishing to be bound by theory, the octapeptide region of prion proteins is believed to bind copper [Cu(II)], which may induce a defined structure to the otherwise random loop structure. The physiological role of prion protein is thought to be copper transport into the cell. Viles et al, Proc. Natl. Acad. Sci. USA 96:2041, 1999.

In some embodiments, a peptide ligand according to the invention binds the prion polypeptide in the presence of 1 nM to 500 μM copper, e.g., 10 nM to 400 μM, 100 nM to 300 μM, or 500 nM to about 100 μM copper.

In one specific embodiment, sequences of an a prion protein, fragment, derivative or analog thereof are modified to include a detectable (e.g. radioactive or fluorescent)label.

If desired, two or more peptide ligands according to the invention can be present in multiple copies. Identical copies of one or more peptides can be present (e.g., homodimers, homotrimers, etc.), or multiple copies of peptides varying in sequence can be present in a ligand (e.g., heterodimers, heterotrimers, etc.).

Use of Ligands to Detect and Remove Prions

Ligands that bind to prion proteins and fragments are useful for a variety of analytical, preparative, and diagnostic applications. In one embodiment, prion ligands can be used to detect the presence of prion protein in a solution sample. In some embodiments, the ligands can be coupled to a solid support, such as a resin or a membrane, and used to bind and detect targets that are present in the solution, e.g., in a biological fluid. See, e.g., Doyle, U.S. Pat. No. 5,750,344. Examples of biological fluids include, e.g., blood, blood derived compositions or serum Additional biological fluids include cerebrospinal fluid, urine, saliva, milk, ductal fluid, tears, or semen.

As used herein, the terms “blood-derived compositions” and “blood compositions” are used interchangeably and are meant to include whole blood, blood plasma, blood plasma fractions, blood plasma precipitate (e.g., cryoprecipitate, ethanol precipitate or polyethylene glycol precipitate), blood plasma supernatant (e.g., cryosupernatant, ethanol supernatant or polyethylene glycol supernatant), solvent/detergent (SD) plasma, platelets, intravenous immunoglobulin (IVIG), IgM, purified coagulation factor concentrate, fibrinogen concentrate, or various other compositions which are derived from human or animal. Blood-derived compositions also include purified coagulation factor concentrates (e.g., factor VIII concentrate, factor IX concentrate, fibrinogen concentrate, and the like) prepared by any of various methods common in the art including ion exchange, affinity, gel permeation, and/or hydrophobic chromatography or by differential precipitation.

In another embodiment, ligands that bind to prion proteins and fragments can be used to detect targets extracted into solution from a solid sample. For example, a solid sample can be extracted with an aqueous or an organic solvent and the resultant supernatant can be contacted with the ligand. Examples of solid samples include plant products, particularly plant products which have been exposed to agents that transmit prions. For example, hay mites have been reported to transmit prions. Accordingly, methods described herein can be used to detect prions in solid samples such as grass and hay. Ligands of the present invention can also be used in some embodiments to detect the presence of prion in soil. Other solid samples can include brain tissue, corneal tissue, fecal matter, bone meal, beef, beef by-products, sheep, sheep by-products, deer, deer by-products, elk, and elk by-products.

Alternatively, or in addition, binding can be used to selectively remove the cognate target or targets from the solution sample or sample supernatant. For selective detection and removal of targets from solutions the ligands can be attached to a solid supports, such as a resin. Resins for removing agents from fluids such as blood or blood compositions are well known in the art and are described in, e.g., Horowitz et al., U.S. Pat. No. 5,541,294; Buettner et al., U.S. Pat. No. 5,723,579; Buettner, U.S. Pat. No. 5,834,318. In one embodiment, the resin is a polymethacrylate resin.

Methods of Identifying Ligands for Prion Proteins

The invention also provides a method of identifying a ligand for a prion protein. The method includes contacting a test agent with a peptide that includes at least three continuous amino acids of the sequence GWGQPHGGGWGQPHGG (SEQ ID NO:2) or three continuous amino acids of the retro-inverso sequence D(GGHPQGWGGGHPQGWG) (SEQ ID NO:34). Formation of a complex between the test agent and said polypeptide indicates the test agent is a ligand for a prion protein.

A “prion protein” may be a “normal” prion protein, also referred to as a “protease sensitive” or “sensitive” prion protein, designated PrPc. The prion protein in the infectious form is called “resistant” or scrapie form, and is designated as “PrPres” or “PrPsc” protein, respectively. Additional ligands that may be detected are those that bind to variants of both the sensitive and resistant forms. The isolates or strains of prion may vary by structure or conformation or by characteristic incubation times of the disease, disease length and pathology. The amino acid sequences of the variants may differ by one or more amino acids.

The test agent (e.g., a ligand) can be, e.g., a polypeptide, peptide, peptidomimetic, small organic molecule, small inorganic molecule, nucleic acid, lipid, or a carbohydrate. Test agents can be monomeric or polymeric compounds. In some embodiments, test agents contain aromatic groups such as a porphyrin ring, a phthalocyanine, a napthoquinone, an imidazole, a purine, or a pyrimidine.

In some embodiments, the peptide includes 4, 5, 6, or 7 continuous amino acids of the sequence GWGQPHGGGWGQPHGG (SEQ ID NO:2) or of the sequence D(GGHPQGWGGGHPQGWG) (SEQ ID NO:34).

An example of a suitable peptide to use in the screening method is one including the octapeptide repeat GWGQPHGGGWGQPHGG (SEQ ID NO:2), and which is amidated at the carboxyl terminus and acetylated at the amino terminal with [¹⁴C] acetic anhydride. This peptide represents a dimer of the octapeptide, and is represented as *acetylGWGQPHGGGWGQPHGGamide.

Other peptides useful for screening for prion ligands include permutations of the repeated sequence such as *acetylPHGGGWGQPHGGGWGQamide (SEQ ID NO:33). Because the sequence GWGQPHGG (SEQ ID NO:1) is repeated at least 4 times in the human prion protein, it is thought that ligands that bind a peptide having multiple repeats binds to the prion protein with increased affinity. However, a single octapeptide repeat GWGQPHGG (SEQ ID NO: 1) can be also used for ligand screening, as can fragments of the sequence, for example, HGGGW (SEQ ID NO:31) or the copper binding motif HGGG (SEQ ID NO:32).

While not wishing to be bound by theory, it is thought that peptide dimers theoretically permit chelation with 2 moles of copper. Viles et al, Proc. Natl. Acad. Sci. USA 96:2041, 1999; Miura, et al., Biochem. 38: 11560 (1999). For the peptide G₁WGQP₅HGGGW₁₀GQPHGG₁₆amide (SEQ ID NO:2), one copper is thought to bind to H₆ and the nitrogen of the peptide bonds between G₇ and G₈ and between G₈ and G₉. A second copper may coordinate to H₁₄, the peptide bond between G₁₅ and Q₁₆, and the nitrogen of the amide bond. For the peptide P₁HGGG₅WGQPH₁₀GGGWGQ₁₆amide (SEQ ID NO:33), one copper is thought to bind to the first H₂ plus the nitrogen of the peptide bonds between G₃ and G₄ and between G₄ and G₅, and the second copper may coordinate to H₁₀ and the peptide bonds between G₁₁ and G₁₂ and between G₁₂ and G₁₃. Accordingly, in some embodiments, the screen is performed in the presence of a metal, such as copper. Typical copper concentrations include a range of 100 nM to about 500 μM, preferably from about 500 nM to about 100 μM.

In one embodiment, a radiolabelled peptide is used in the ligand screening. For example, a radiolabelled peptide containing some or all of an octapeptide-repeat derived sequence can be chemically synthesized and screened for the ability to bind to ligands in synthetic combinatorial library. Complexes of the peptide and members of the library can be identified using the radiolabelled peptide.

If desired, a ligand library can be used in the screening methods. As used herein, a ligand library means at least two, (e.g., 5; 10; 50; 100; 200; 500; 1,000; 2,500; 5,000; 10,000; 25,000; or more) molecular entities with different sequences. Libraries can include polymeric ligands such as nucleic acids, carbohydrates, or peptides. In the case of peptide libraries, the amino acid building blocks can be the 20 genetically encoded L-amino acids, D-amino acids, synthetic amino acids, amino acids with side chain modifications such as sulfate groups, phosphate groups, carbohydrate moieties, etc. A random peptide library may include a mixture of peptides ranging in length from 2-100 amino acids or more in length, but are typically about 5-15 amino acids in length. The term “random” indicates only the most typical preparation of the library, and does not require that the composition be unknown. Thus, one may prepare a mixture of precisely known composition if desired. The libraries can also include non-oligomeric ligands, e.g., small non-oligomeric organic ligands, including aromatic ligands such as porphyrin rings, phthalocyanines, napthoquinones and imidazoles.

For screening of peptide ligands, either biologically derived (e.g. phage) or chemically synthesized combinatorial libraries can be used. The latter is generally preferred since it is faster to generate and has greater diversity, as it can utilize non-natural amino acids. The members of the combinatorial structures can be generated on the surface of an inert support such as a chromatography bead or a silicon chip. Chromatography beads can be e.g., 100 um in diameter. Each bead has a single structure synthesized on its surface and each bead may have a unique structure. Therefore, 1 ml of resin, which contains over 1,000,000 beads may have a comparable number of unique sequences.

In general, any art-recognized method for constructing a ligand library can be used. The development of synthetic peptide combinatorial libraries on inert surfaces has made available large numbers of distinct peptides for studying ligand-target interactions. Random peptide libraries can be produced by standard organic synthesis of amino acids polymerized on micro beads. Typically, the peptides on any one bead in a library are substantially the same; however, the peptide sequences vary from bead to bead. For example, a mix, divide and couple synthesis method can be used generate unique peptide sequences on polystyrene-based resinous beads, as described in Furka et al., Int. J. Pept. Protein Res. 37: 487-493 (1991); Lam et al., Nature 354: 82-84 (1991). Surface-bound, chemically synthesized libraries can now be purchased from commercial vendors. For example, a peptide library can be obtained from Peptides International, Inc. (Louisville, Ky.).

A library of ligands can be affixed to a surface (e.g., bead) using any attachment method which results in a linkage that is stable enough that the relative spatial locations of members of the library can be detected on the surface.

In some embodiments, a library containing peptide ligands is constructed by synthesizing peptides on a polymethacrylate resin. For example, a reactive group on the resin is the site of attachment of a first protected amino acid that is coupled through its carboxyl group. Following coupling, the protected amino group of the first amino acid is deprotected to expose a new amino terminus. This new amino terminus functions as a site for attachment of the next protected amino acid. Through cycles of coupling and deprotection the library is grown from the initial reactive group of a resin, such as TentaGel resin (Peptides International, Inc., Louisville, Ky.).

In some embodiments, individual beads, each carrying a unique ligand, are immobilized on a surface. In other embodiments, the resin containing the ligand library is placed in a column, or screened in batch format.

Since a large number of ligands can be synthesized onto the surface of beads, it is possible to produce enormous volumes of beads—most of which have a unique ligand. However, synthesizing vast numbers of, for example, peptide ligands and screening every bead for binding to a target is technically difficult. Thus, one method of selecting a candidate ligand involves screening smaller libraries for binding activity. Once a lead has been found additional ligands (sub-libraries) are synthesized based on the lead ligand. Screening of these sub-libraries may lead to additional and improved leads being discovered. Through a process of iteration of synthesis and screening it is possible to identify preferred ligands.

Any detectable difference between non-bound ligands and ligands bound to targets can be exploited. For example, a probe molecule which recognizes the prion target can be added to the screened ligand library and allowed to bind. Alternatively (or in addition), a fluorescently labeled prion target bound to ligands can be detected spectrophotometrically. In certain embodiments, the target is not itself labeled, but after the binding reaction, the target is reacted to produce a detectable signal (i.e., a light emitting signal). Radioactively labeled molecules may also be used to detect the presence of a ligand-prion complex. For example the prion target can be labeled with radioactive ¹⁴C, ³⁵S or ¹²⁵I. In other embodiments, antibodies to the prion target are used as detection molecules. Carbonell, et al., have developed a screening method that includes radiolabelling a target (U.S. Pat. No. 5,783,663; Bastek, et al., Separation Science and Tech., in press, 2000), exposing the target to a combinatorial library, washing the beads with large volumes of buffer, plating the beads in agar and detecting the beads that bind to the radiolabelled target by autoradiography film.

In some embodiments, ligands are identified in the presence of plasma. This can be accomplished, for example, by adding blocking solutions to the beads of the library, then plasma, if desired, and finally adding the radiolabelled peptide to the plasma. After incubation, the beads are placed in a column and washed until all non-bound radioactivity is removed. The beads are then plated in soft agar and after drying are overlaid with autoradiography film.

Once a ligand-prion target complex has been detected, the bead is isolated and the ligand is identified and further characterized. Identifying the ligand may include re-screening some members in an original library from a region of a ligand library containing putative target-binding ligand. This will typically be performed when the ligand library is plated at a relatively high density.

For example, if the ligand is a peptide, it is microsequenced by Edman degradation. The decoded sequence is then synthesized on a resin. Binding of the radiolabelled peptide is confirmed by exposing this second resin to radiolabelled prion peptide and recovering the non-bound radiolabel following separation by centrifugation and filtration.

To confirm that a test agent is a ligand for a prion-derived peptide (i.e., the octapeptide), secondary tests for octapeptide binding to ligand can be performed using methods known in the art, such as equilibrium binding. The matrix in which the studies are being undertaken may influence the relative affinity of binding of a peptide or protein to ligand under the experimental conditions. One preferred matrix is phosphate buffered saline (PBS).

Methods of Detecting Prion Proteins

The invention also provides a method of detecting the presence of a prion protein in a biological fluid. The biological fluid, e.g., a test sample, is contacted with a ligand according to the invention under conditions sufficient to cause formation of a complex between the prion protein, if present, in the biological fluid and the ligand. The complex is then detected, thereby detecting the presence of the prion protein in the biological fluid.

The presence of a prion protein can be examined in any desired biological fluid. Suitable biological fluids will typically include, e.g., blood, blood fractions and compositions, cerebrospinal fluid, urine, saliva, milk, ductal fluid, tears, and semen.

An additional approach to demonstrate the ability of the sequences identified by screening to bind the entire prion protein is to add a number of beads,(e.g., about 200) to each of a number of wells of a 96-well micro-titer plate. Brain extracts containing prion are then added to the beads and, following incubation, the non-bound prion is removed by repeated washing. The presence of prion is then detected by adding prion specific antibodies, phosphatase conjugated secondary antibodies and phosphatase substrate. Additional control wells define the amount of signal due to non-specific binding, endogenous phosphatase, non-specific antibody binding etc. This format may be developed to form a large throughput assay for identifying prion protein. The method of detection of prion protein provided by the present invention may be used for detecting prion protein in many different types of samples. For example, the prion-containing sample may be a liquid bodily fluid such as blood, blood products or fractions (e.g., plasma and serum), cerebral fluid, urine, saliva, ductal fluid, tears, semen, water or milk. The sample can also be the supernatant derived from the extraction of a solid sample such as brain tissue, corneal tissue, fecal matter, bone meal, beef, beef by-products, sheep by products, deer, deer by products, elk, elk by products, soil, hay, or animal feed. Because BSE is transmitted to cows through foodstuff supplemented with beef by-products, a method for detecting prion proteins in material such as foodstuffs is extremely useful.

Methods of Removing Prions from Biological Fluids

Also included in the invention is a method of removing a prion from a biological fluid. The method includes contacting the biological fluid with a prion-binding ligand according to the invention under conditions sufficient to cause formation of a complex between the prion protein, if present, in the biological fluid and the ligand. The complex is removed from the biological fluid, thereby removing the prion from the biological fluid.

The prion protein can be removed from any desired biological fluid. Suitable biological fluids will typically include, blood, blood products or fractions (e.g., plasma and serum), cerebral fluid, urine, saliva, ductal fluid, tears, semen, water or milk.

Prion proteins may also be separated from other proteins in a sample by using affinity chromatography. In this instance, the ligand or agent according to the invention which binds a prion protein or peptide is coupled to a solid support, e.g. an inert support such as a membrane or resin, and the prion protein binds to the immobilized agent. If desired, one of the sequences obtained from the initial screening is immobilized on a resin, such as polymethacrylate. Other types of resin that may be used include, e.g., sepharose, cross-linked agarose, composite cross-linked polysaccharides, celite, acrylate, polystyrene and cellulose. Membranes such as, for example, nylon and cellulose may also be used.

Elution of prion protein from the ligands is influenced by the affinity of the prion for the ligand. Prions exist in different conformational states and different degrees of aggregation. Infectious prion is frequently aggregated and will thus be expected to have higher affinity for the resin than the non-aggregated non-infectious prion protein. This is due to the likelihood of multiple interactions between the different molecules of the aggregate and the ligands on the resin. This higher affinity may manifest itself in a different elution profile for PrPc compared to PrPsc. Interactions between prion protein and the ligands determined from screening of the octapeptide repeat peptide may be influenced by the structure of the prion protein adjacent to the actual binding sequence. In addition, ligands may participate in multiple interactions with other sites on the prion protein itself. These are expected to be different between PrPc and PrPsc. Thus, although all mammalian prion proteins that contain the octapeptide repeat will bind to the octapeptide specific ligands the overall binding affinity of the protein to the ligand will likely vary for different prions. The elution profile of prion proteins will be influenced by the fact that prion protein binds prion protein. Thus, the strength of interaction will likely be enhanced by direct prion-prion interaction on the surface of the resin.

Methods of Treating or Preventing Prion-Associated Pathologies

The ligands according to the invention can be used in a method of treating or retarding the development of a prion-associated pathology in a subject. The method includes administering to the subject a ligand of the invention in an amount sufficient to treat or retard the development of the pathology. In some embodiments, the subject is a mammal, e.g., a human, cow, horse, dog, cat, rat, mouse, or deer.

Prion-associated pathologies which can be treated include those in which prions, or prion-related agents, have been implicated as causative agents. These conditions include e.g., Creutzfeldt-Jakob disease (including iatrogenic, new variant, familial, or sporadic forms) Gerstmann-Straussler-Scheinker disease, fatal familial insomnia, scrapie, bovine spongiform encephalopathy, transmissible mink encephalopathy, feline spongiform encephalopathy, exotic ungulate encephalopathy and chronic wasting disease.

A ligand of the invention may be administered intrathecally (IT), intracerebrovertricularly (ICV) or systemically, for example, intraperitoneally (IP). Solubility of the ligands may be enhanced by admixture with a solubilizing agent, for example, cyclodextran. In an alternative embodiment, a ligand according to the invention, is administered in conjunction with one or more additional agents for treating or preventing a prion-related pathology.

Pharmaceutical Compositions

The ligands of the invention can be incorporated into pharmaceutical compositions suitable for administration. Such compositions typically include the ligand and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the ligand of the present invention to affect solubility or clearance of the ligand.

If necessary, the ligands can be co-administered with a solubilizing agent, such as cyclodextran.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycol, glycerol, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The composition is preferably sterile and should be fluid to the extent that easy syringability exists. It is preferably stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ligand according to the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the ligands are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

If desired, oral or parenteral compositions can be prepared in dosage unit form for ease of administration and uniformity of dosage. Dosage unit form as used herein refers to physically discrete units suited as unitary dosages for the subject to be treated; each unit containing a predetermined quantity of ligand calculated to produce the desired therapeutic effect in association with the required pharmaceutical carrier. The specification for the dosage unit forms of the invention are dictated by and directly dependent on the unique characteristics of the ligand and the particular therapeutic effect to be achieved, and the limitations inherent in the art of compounding such an active compound for the treatment of individuals.

Nucleic acid molecules encoding peptide ligands of the invention can be inserted into vectors and used as gene therapy vectors. Gene therapy vectors can be delivered to a subject by, for example, intravenous injection, local administration (see U.S. Pat. No. 5,328,470) or by stereotactic injection. ee e.g., Chen et al. PNAS 91:3054-3057(1994)). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

The following Examples are presented in order to more fully illustrate the preferred embodiments of the invention. These Examples do not limit the scope of the invention, as defined by the appended claims.

### EXAMPLES

### Example 1

### General Methods

Prion Peptide

The peptide *acetylGWGQPHGGGWGQPHGGamide (SEQ ID NO:2) was prepared at a specific radioactivity of 11.2 mCi/mmol and was synthesized by Commonwealth Biotechnologies of Richmond, Va. In brief, the full length peptide was synthesized, deprotected and the carboxyl terminal amidated. The peptide was purified by reverse phase HPLC and the amino terminal was acetylated with radioactive acetic anhydride. The peptide had a purity of greater than 95%.

Peptide Synthesis on Beads

Peptide ligands were synthesized directly onto Toyopearl 650 M amino resin from TosoHaas (Buettner et al., Int. J. Pept. Protein Res.47:70, 1996) by means of standard Fmoc peptide chemistry. A single epsilon amino caproic acid (‘eaca’) was first conjugated on the amino group of the resin to act as a spacer between the resin and the ligand. The fidelity of synthesis and the density of the peptides was determined by amino acid analysis. These values typically ranged from 30 to 200 μmol/gram dry weight. Following deprotection of the side groups some samples of resin were acetylated at the amino terminal. Confirmation that acetylation was complete was determined by the absence of ninhydrin staining (Methods in Molecular Biology, Humana Press 1994).

Peptide Libraries

Peptide libraries were synthesized on Toyopearl resin according to Buettner, et al., Int. J. Peptide Protein Res. 47:70-83 (1996). Libraries were either synthesized at full density about 200-300 μmol/gram dry weight or were synthesized with decreased density 50 μmol-100 μmol/gram dry weight by partial blocking of the amino groups with tBoc amino acids prior to synthesis of the peptide. The low density library had a ligand density of about 10-20 pmol per bead.

Additional Peptides

Other peptides used during this study included peptide 76, acetyl (dF)LLHPI (SEQ ID NO:35), peptide 55, HHHPQT (SEQ ID NO:36), which appears to bind prion only in the absence of copper, and peptide 71, RYHVYF (SEQ ID NO:37).

Library Binding Studies

The contacting of the radiolabelled octapeptide was performed according to the following strategy. Non-specific binding of protein/peptides to about 30 mg (20,000 beads) of library was minimized by incubation in 1% casein for 2 hours. The majority of the casein was then removed. The beads were then contacted with solvent detergent treated plasma (VITEX Licensed Facility, Melville, N.Y. 11747) for 2 hours to allow for additional blocking of non-specific binding sites on the library. Solvent-Detergent plasma is a plasma composition that has been treated with an organic solvent and a detergent to remove blood-borne lipid coated viruses. Many different reagents used to accomplish this have been proposed in the art. See, e.g., U.S. Pat. Nos. 4,481,189; 4,540,573; 4,764,369; 4,789,545, all incorporated herein by reference. The most preferred reagents of this invention include an effective amount of an alkylphosphate reagent, preferably tri-(n-butyl) phosphate (TNBP) as the “solvent” and Triton X-100 as the detergent. Treatment of a sample with these reagents is followed by extraction with soybean oil followed by column chromatography on a standard hydrophobic resin to remove the virus-inactivating reagents. These methods provide viral-inactivated blood-derived compositions without denaturing the indigenous proteins.

The peptide was then added to the blocked beads at a final concentration of 20 μM, 40 μM, or 50 μM (for the low-density library). After a 1-5 hour incubation, the beads were transferred to a column (BioRad, PolyPrep) and washed with PBS (pH 7.4) with Tween 20 at 0.05% (W/V). Samples of the eluate were taken and counted for radioactivity. After the eluate counts had decreased to background levels (<50 dpm) the beads were removed from the column and suspended in 90 ml of 1% agarose warmed to about 45° C. The agarose was layered as a thin film on 2 films of Gel Bond films from FMC Bioproducts Cat. No. 53749 and allowed to dry at room temperature. Following drying an autoradiography film (Kodak, Biomax MR1) was placed directly on the agarose and was exposed for several days. The film was developed and a few spots were identified. The film was aligned with the beads and those generating the signal were excised. Usually, 2 films were developed for each experiment to ensure that the signals were true and to help ensure that the correct bead was identified.

Prion

Brain homogenates were prepared from scrapie infected hamsters. In brief, brains from hamsters infected with scrapie were isolated, homogenized in PBS (at 10% W/V). The cell debris was removed by low speed centrifugation, and the supernatant was used. The extracts of hamster brains were provided by Robert Rohwer at the VA Medical Center Medical Research Service (Baltimore, Md.). In some embodiments, the brain homogenate was solubilized in 10% sarkosyl.

Prion Detection

PrP was detected using monoclonal antibodies, including “3F4”, available from Senetek PLC (St. Louis Mo. 63128). Antibody 3F4 recognizes the peptide sequence MKHM (amino acids 109-112, SEQ ID NO:39) in the native conformation of PrPc and also MKHM (SEQ ID NO:39) of the denatured, but not the “fibril” or aggregated form of PrPsc. Consequently, in order to detect the presence of aggregates of PrPsc using 3F4, the PrPsc must first be denatured. Other monoclonal antibodies used for detection of prion include FH11 which binds to both PrPc and PrPsc at, or near the octapeptide repeat. Streptavidin binds the sequence HPQ through interaction of the side groups of the H and Q and binds to resins containing the peptide GWGQPHGG (SEQ ID NO:1) and it has been proposed that it may also be useful for the detection of prion protein (WO 00/02575) through binding to the octapeptide repeat.

Prion Binding

The screen for binding normal brain prion was performed using 96 well microtiter plates (Cat. No.3075 from Falcon, Becton Dickinson). The plates were first blocked with 100 μl/well of 1% (W/V) casein from Pierce at 65° C. for 1 hour. Dry resin was swollen in ddH₂O. The well was emptied and 30 μl of a suspension of swollen resin (total about 200 beads) was added to the well. Resin was allowed to settle and surplus water was removed. Various additions of source material and reagents were evaluated. Under these experimental conditions, 50 μl of normal brain homogenate was diluted 1:3 in 5% human serum albumin from Alpha Therapeutic Corp and was heated at 65° C. for 1 hour to inactivate residual phosphatase before use. The brain/albumin preparation was incubated at room temperature for 1.5 hours with the beads after which time the non-bound protein solution was removed and 100 μl of 3F4 monoclonal antibody (Senetek, Cat. No. 620-02) diluted 1:1000 in 1.0 % casein was added into the experimental wells. The beads were incubated with primary antibody overnight at 4° C. with gentle agitation. Control wells had 100 μl of 1% casein, with no antibody as appropriate. The beads were then washed once with PBS+10 μM CuCl₂ at pH 7.4. The secondary antibody, anti-mouse IgG alkaline phosphatase (AKP) conjugate (product # A-3688 from Sigma) was diluted 1:1000 in 1% casein. Incubation proceeded for 45 minutes at room temperature. At this time it was washed 2× in PBS+Cu+Tween 20 (0.05%) at pH 7.4, 2× in PBS+Cu, 2× in 1M NaCl, once in NaHCO₃+5 mM MgCl₂ at pH 9.5. Substrate (100 μl of CDP Star/well diluted 1:100 in NaHCO₃+MgCl₂ (CDP Star cat # 6209 from Tropix) was then added.

A filter paper (cat # 1703932 BioRad was cut to shape and wetted with NaHCO₃+MgCl₂. 25 μl of bead suspension was added per dot on the filter paper. The filter paper was wrapped in seal wrap and exposed in a cassette to autoradiography film. Different pieces of film were developed at different time points typically ranging form 1 to 10 min.

Column Binding

A 1:10 dilution of PrPsc brain homogenate was treated with sarcosyl at a concentration of 20 μl per 4 ml of brain and was incubated for 30 min and centrifuged. The supernatant was carefully removed and diluted in PBS or plasma as appropriate. 0.5 ml of resin was added to a chromatography column. The prion solution was allowed to contact the resin for 20 min at room temperature before 2×1 ml aliquots of PBS+10 μM CuCl₂ was added. The flow through was recovered. In some experiments the resin was contacted with 5 column volumes of 100% plasma before a 50% solution of prion in plasma was added.

Fractions were either dissolved in SDS-PAGE buffer or were first treated with 1 mg/ml proteinase K in 50 mM Tris, 10 mM NaCl, 2 mM CaCl₂ for 1 hour at 37° C. The final proteinase K activity was 10 μg/120 μl. When plasma was present, the incubation time was extended to 1.5 hours. At this time 5 μl of 22 mg of PMSF (Sigma, St. Louis, Mo.) in 0.5 ml of methanol was added. The samples were heated at approximately 100° C. for 10 min before SDS was added to a final concentration of 2% and boiled again for 10 min. Samples were then prepared in sample buffer containing 0.05% dithiothreitol and were heated again at approximately 100° C. for 3 min.

Western Blots

Protein samples were rigorously dissolved in SDS-PAGE buffer from Novex (Tris-Glycine SDS Sample buffer Cat.# LC2676) and heated at >95° C. for 3 min. 15 μl samples were loaded onto 14% gels (Novex Cat.# EC6485) and electrophoresed at a voltage of 125V for 90 min. At this time the protein was transferred to PVDF membranes (Novex 0.2 μm Pore Size Cat #.LC2002), previously soaked in 100% methanol. The proteins were transferred for 1 hr at 250 mA. The membranes were blocked with 5% non-fat dried milk in TBST (50 mM Tris, 0.15M NaCl +0.05% Tween 20 pH 7.4) at room temperature for 1 hour. At which time a 1:1,000 dilution of 3F4 from Senetek in 5% non-fat dried milk was added. Incubation was from 1-16 hours at which time the primary antibody was removed. The membrane was washed 3× in PBST before the secondary antibody was added. Secondary antibody (either sheep anti-mouse horse radish peroxidase from Amersham or phosphatase conjugated anti-mouse IgG from Sigma was added at a dilution of 1:3000 in 5% non-fat dried milk in TSBT.

Peroxidase and phophatase detection systems were evaluated. The secondary antibody was allowed to bind for 60 min at room temperature and was then washed three times with TBST. The developing reagents were either “ECL +” from Amersham or SuperSignal West Dura Extended Duration Substrate from Pierce Cat. # 34075 or “CP-Star” from Tropix.

### Example 2

### Octapeptide Screening

The results of the sequences obtained from screening of the octapeptide are given in the second column of Table 1. The sequences show a prevalence of aromatic amino acids and have a number of similarities. For example, 12 of 28 have the consensus sequence OxxO where O is an aromatic amino acid and x an amino acid. Other similarities are also found e.g. RWIISL (SEQ ID NO:24) and LRVIIS (SEQ ID NO:15). The occurrence of a consensus sequence around the structure WLYWIP (SEQ ID NO:4) is found in a number of peptides (see Table 2). For example, WLVWLA (SEQ ID NO:27) has four of 6 amino acids exactly the same as WLYWIP (SEQ ID NO:4) as does LLIWIP (SEQ ID NO:3). Other peptides have very similar structures. A number of peptides were obtained following screening in the presence of additional 100 μM CuCl₂ which was added to the plasma and octapeptide prior to incubation and washing with 10 μM copper in the PBS (GibcoBRL, Life Technologies Cat. No.14080-055. These structures are indicated by the inclusion of Cu in the comment column and show the characteristic predominance of aromatic amino acids suggestive that copper binding does not inhibit the interaction of octapeptide with ligand. Binding was also evaluated at low pH (4.5) and was followed by washing at pH 4.5, however, no leads were obtained.

The results of equilibrium binding the radiolabelled octapeptide to the resin are also shown in Table 1. For each sample, 15 mg dry weight of resin was weighed out and swelled in PBS pH 7.4. The resin was washed 5 times with 400 μl of PBS and then washed twice with buffer containing 10 μM CuCl₂. Radiolabelled octapeptide was added to a final concentration of 15 μM. It was incubated for 2 hours at room temperature. The final volume was 400 μl of solution plus 45 μl of resin. At this time the non-bound peptide was removed by centrifugation in a Millipore filter unit (Ultra Free-mc Centrifugal Filter Device Cat. 8UFC30HVNB). The flow through solution was then counted for radioactivity. All peptides were evaluated with a free amino group at the end terminal.

The amount of non-specific binding of the peptide to the experimental set up (e.g., the filter) was assessed to be about 1.5%. The 15 mg of dry resin used in this experiment swells to a weight of about 45 mg, demonstrating that 30 μl of solution was present in the gel. Thus, 30 μl/430 μl, or 7% of the free octapeptide was held up in the gel. Since the total volume of the reaction mix was 430 μl, a theoretical recovery of about 92% is expected if no binding to the resin occurs. This figure is seen for the amino resin. However, the acetylated amino resin does have some binding potential.

The highest affinity binder under the specific conditions employed in this experiment was GFFFWW (SEQ ID NO:8). It is not possible to conclude from a single experiment the affinity of octapeptide for ligand since the stoichiometry of binding is not known, nor is the actual concentration of free ligand. The highest affinity binder has 5 aromatic groups, the second and third best both have 4. There is significant difference between WEFYWF (SEQ ID NO:5) with a result of 76% and GFFFWW (23%) (SEQ ID NO:8) although both have 5 aromatic groups. The binding studies were performed in saline in the presence of 10 μM Cu which would be sufficient to bind only 33% of the total copper binding capacity of the octapeptide which was added at a concentration of 15 μM. However, this condition allowed differentiation of high and lower affinity ligands to be ascertained. The effect of copper on binding was evaluated for the binding of 15 μM octapeptide to GFFFWW (SEQ ID NO:8). In the absence of added copper 92% of the radioactivity flowed through the amino resin whereas only 22% flowed through the GFFFWW (SEQ ID NO:8) resin. At 15 μM CuCl₂ the figures were 92 and 19%, respectively; at 30 μM Cu they were 94 and 18%, respectively, and at 60 μM the numbers are 92 and 17%, respectively. Thus, added copper has little or no effect on the biding of the radioactive prion peptide to the resin alone or to the resin-peptide complex. The lack of observed effect may arise because copper is either not important for binding, or alternatively, it is already bound to the peptide during peptide synthesis or purification. No binding was seen at pH 4.5 for any of the resins.

Table 1 also shows the peptide sequences shown to bind to the radiolabelled octapeptide repeat column. The sequences were selected from either high or low-density libraries. Percentage of binding (%) is the amount of radioactivity that did not bind to the resin using 15 μM starting peptide. Sequences 110 through 119 were screened in the presence of an additional 100 μM CuCl₂, to ensure saturation of the copper binding sites. The consensus column indicates the presence of aromatic “O” and non-aromatic “x” amino acids.

### Example 3

### Prion Binding to Resin

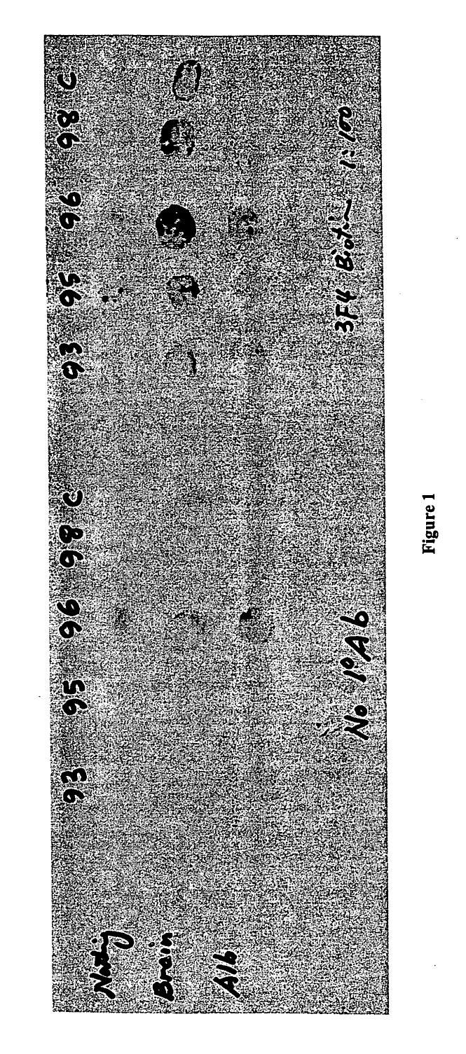

The signals of the autoradiography film for beads with sequences 93: LEIRLA (SEQ ID NO:12), 95: SLEEYV (SEQ ID NO:14), 96: LRVIIS (SEQ ID NO:15), 90/98: FYVFTA (SEQ ID NO:9) and control amino resin are shown in FIG. 1. The samples marked ‘C’ are the control resin with no attached peptide. Each sample contained approximately 200 beads of the same peptide sequence. The left side “No 1° Ab” had no prion specific 3F4 antibody added, while the other five samples were probed with 3F4 prion specific antibody. The top row, labeled ‘Nothing’ was a control containing neither brain extract nor albumin. The second row (‘Brain’) shows the beads that had been contacted with brain extract in albumin. The third row (‘Alb’) shows samples that had only albumin added. All samples had a goat anti-mouse IgG secondary antibody added. After probing, the beads were transferred to filter paper, and the amount of bound prion was determined by chemiluminescence intensity.

The results from peptide 96 indicated that incubation with brain plus secondary antibody 3F4 produces a much larger signal than the controls (normal or albumin). Experiments were duplicated for a number of resins by incubation with extracts of normal brain and probing with 3F4. Extremely similar results were obtained for each of the duplicates, suggesting that this assay is a reproducible method for evaluating prion binding. FIG. 1 also indicates that free amino resin (C) binds some prion protein.

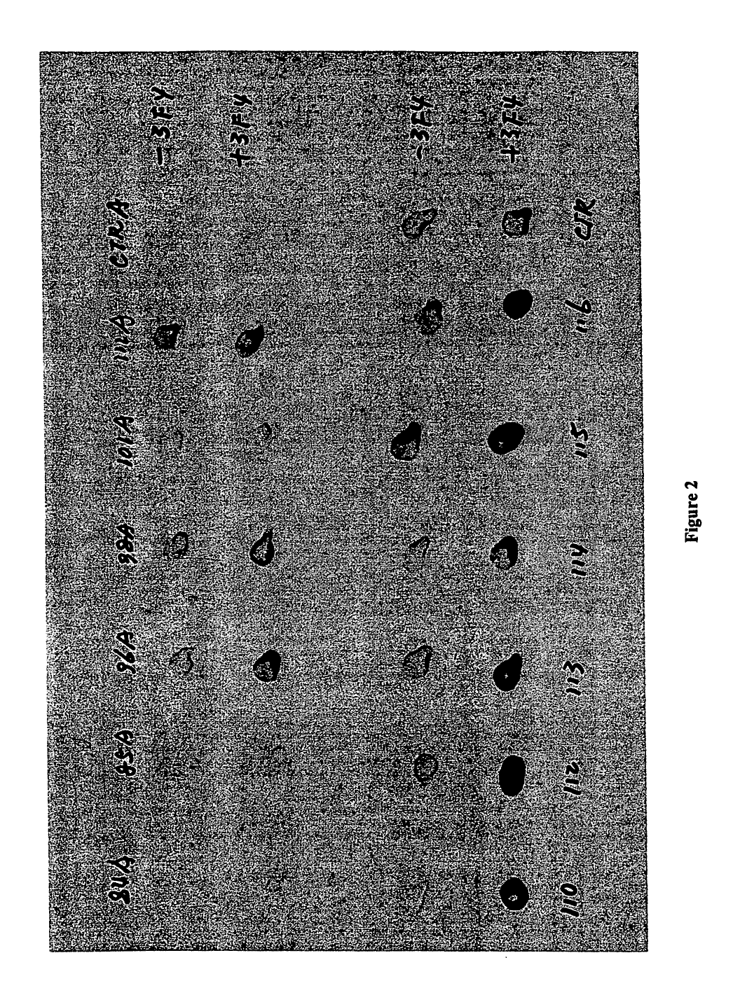

FIG. 2. Demonstrates the secondary binding studies of PrPc from non-infected prion to acetylated resins containing peptides 84, 85, 96, 98, 101, 111, acetylated control resin (top samples), and non-acetylated peptides: 110, 112, 113, 114, 115, 116 and amino control resin (bottom, samples). Normal brain suspended in albumin was added to all wells. The rows marked −3F4 did not have 3F4 added while the rows marked +3F4 included a 3F4 probe. The difference in intensity between +3F4 and −3F4 indicates the amount of PrPc bound to the peptide. The binding of acetylated resins 84 and 85 to prion was not stable enough to resist the washing protocol. The strongest binders in this assay were found to be peptides 110, 112, 113, 115 and 116. Acetylated peptide 110 is uniformly one of the best binders determined by this assay.

The secondary binding studies shown in FIG. 2 confirmed that the amino resin Toyopearl 650 M binds prion, whereas the acetylated resin does not. Binding of prion protein to amino resin may in part be facilitated through ionic interactions with the amino groups, however, the octapeptide itself does not bind to the amino resin. Accordingly, the amino resin itself can be used to bind prion, for example to detect and/or remove prion from a sample, such as a biological fluid or an environmental sample.

### Example 4

### Removal of Prion Protein

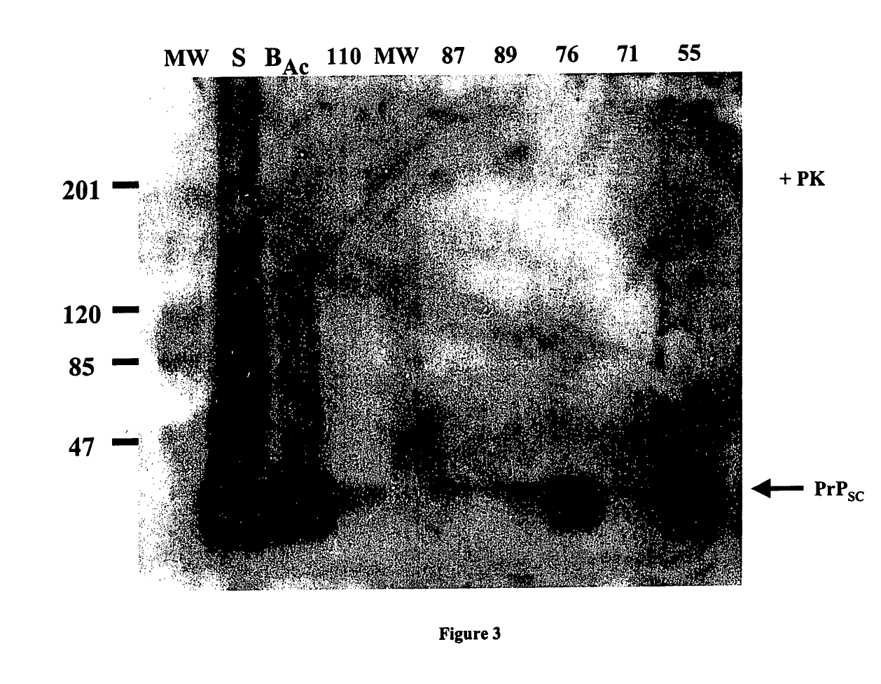

Removal of prion from homogenates of scrapie infected hamster brains is seen in FIG. 3, which depicts a Western blot of the binding of prion from infected brain to various peptide resins. In this experiment the peptide resins were acetylated at the amino terminals and contacted with infected brain for 20 min. The flow through was collected and analyzed. Prion adsorbed onto the column was removed by suspension in SDS-PAGE buffer and subsequently evaluated by Western blot. Molecular weight markers were included in lanes 1 and 5 (MW). The undiluted starting material is present in lane 2. The acetylated resin (lane 3, B_{AC}) shows strong bands indicative of prion protein present in the flow-through, indicating minimal binding of the prion to the control resin. Lane 4 shows the lack of a prion signal in the flow through of peptide 110, indicating that the protein bound to the column. Peptide 87 (lane 6) and peptide 89 (lane 7) bound all prion, as evidenced by the lack of a protein signal in the flow through. Peptide 76 (acetyl (dF)LLHPI, SEQ ID NO:35) failed to bind all prion. Peptide 71 (a positive control: acetyl (dR)YHVYF, SEQ ID NO:37) bound all prion while peptide 55 (acetylHHHPQT, SEQ ID NO:36) let prion flow through. Proteinase K digests of the extracts provided identical conclusions.

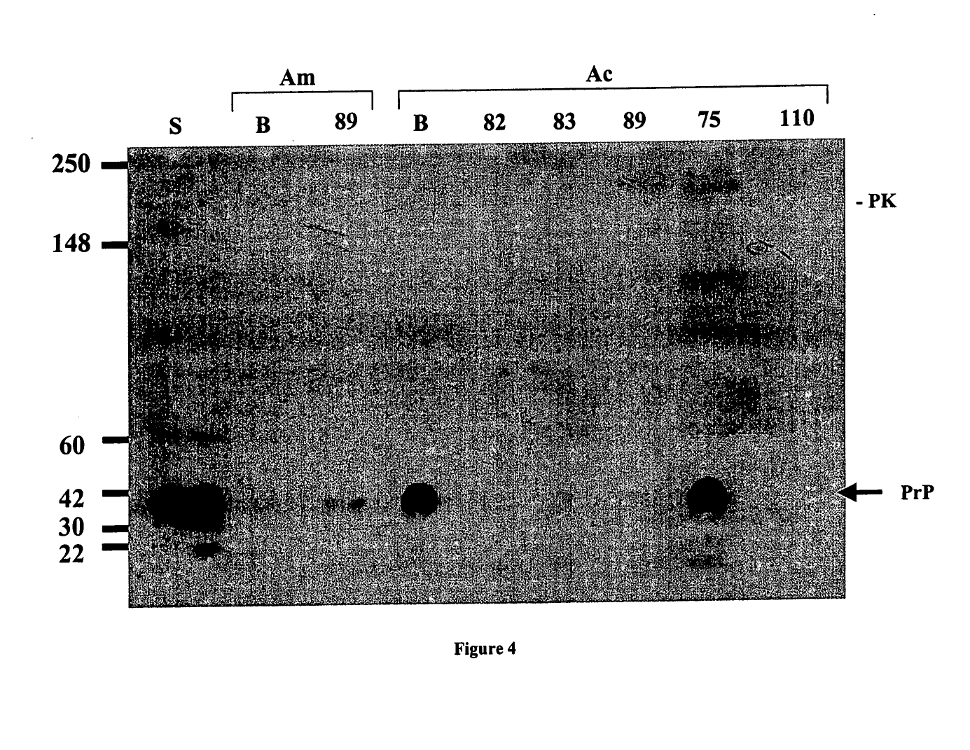

FIG. 4. depicts a Western blot showing the binding of prion from infected brain that had been spiked into plasma. The peptide resins were acetylated at the amino terminals and then contacted with plasma containing infected hamster brain. The resins were contacted with 5 column volumes of plasma prior to analysis of prion binding. The plasma wash blocked any non-specific binding sites. Infected brain homogenates were then diluted 50:50 with plasma and applied to the resin. The flow through from each resin was analyzed for the presence of prion protein. The non-bound material was collected and analyzed. Lane 1 contains the starting material, lane 2 is the amino resin control, lane 3 is amino-peptide 89, lane 4 is the acetylated base resin control, lane 5 contains the flow through for resin containing peptide 82, lane 6 contains the flow through for resin containing peptide acetylated (dR)YHVYF (83) (SEQ ID NO:37), lane 7 contains the flow through for resin containing peptide 89, lane 8 contains the flow through for resin containing peptide acetyl (dF)LLHPI (75) (SEQ ID NO:35) and lane 9 contains the flow through for resin containing peptide 110.

The results in FIG. 4 indicate that the acetylated resin (Ac B) and the control resin containing peptide 75 (acetyl (dF)LLHPI, SEQ ID NO:35) did not bind prion strongly enough to retain all detectable protein on the resin. The positive controls containing peptides 82 and 83 bound all prion, as did peptides 89 and 110. Amino resin 89 failed to bind 100% prion as indicated by the small amount of signal present in the flow through. In this experiment the amino resin (Am B, lane 2) bound all detectable prion. Because acetylated peptides 89 and 110 did not contain any charged amino acids, they cannot be interacting with the prion through ionic interactions. Peptide 75, acetyl (dF)LLHPI (SEQ ID NO:35) is largely hydrophobic, but was not an efficient binder of prion. Thus, a hydrophobic peptide sequence alone is insufficient for effective binding of prion protein.

Taken together, FIGS. 3 and 4 demonstrate that the peptides generated to the octapeptide repeat do indeed bind to the octapeptide and also bind to the prion proteins PrPc and PrPsc.

The octapeptide repeat sequence presents an attractive target for targeting affinity ligands. This is because it is present in multiple copies in all known mammalian prions, it is selective for the prion protein, is accessible for both infectious and non-infectious prions. The octapeptide forms a defined structure in the presence of physiological amounts of copper. Importantly, the octapeptide is believed to be important for infectivity. Thus, prions lacking the octapeptide repeat are not likely to be either non-infectious or may have reduced virulence compared the full length PrPsc.

Screening for binders to the octapeptide repeat revealed that only a small percentage of beads: approximately one in 20,000 bound the octapeptide. This indicates a high degree of selectivity. Following sequencing of the beads a number of different consensus sequences emerged suggesting that the octapeptide could form a number of structures that could be detected by families of related structures. For example many had two (or more) aromatic amino acids separated by two other amino acids. Aromatic structures such as Congo red, porphyrins or phthalocyanines are known to interact with prions though their mode of interaction is not known. It is possible that these may also target the octapeptide repeat. Infected prion forms plaques, which can be stained by Congo red suggesting even plaque, is accessible to aromatic ligands.

The affinity of certain ligands to the prion was different in the two assay systems employed: binding of octapeptide to ligand and binding of prion protein to ligand. It is possible to account for this difference by the octapeptide repeat structure being influenced in the whole protein by the presence of adjacent amino acids or the presence of multiple interactions which may provide affinity or repulsion of the ligand for different structures of on the surface of the prion protein.

The octapeptide repeat might be expected to bind chelators of copper such as histidine. However, only a total of 4 histidines were present in all 28 sequences. Random chance would suggest about 9 (28 total amino acids×6 amino acids per peptide /18 different amino acids) be present. When compared to the number of phenylalanines (21), tryptophans (24) and tyrosines (15) and isoleucines (25) the number of histidines is very low. A number of other amino acids were present at low levels, e.g. aspartic acid was not found. These observations demonstrate that the selection of sequences in the prion binding peptide ligands is not random. This is further established by the generation of consensus sequences.

TABLE 2
Consensus sequence based on the structure of WLYWIP (SEQ ID NO:4)

2^{nd}

| | | | | | |
|---|---|---|---|---|---|
| 1^{st} amino | amino | 3^{rd} amino | 4^{th} amino | 5^{th} amino | 6^{th} amino |

| | | | | | | |
|---|---|---|---|---|---|---|
| acid | acid | acid | acid | acid | acid | SEQ ID NO:4 |

| | | | | | | |
|---|---|---|---|---|---|---|
| L | L | I | W | I | P | SEQ ID NO:3 |
| W | L | Y | W | I | P | SEQ ID NO:4 |
| W | L | V | W | I | A | SEQ ID NO:27 |
| I | Q | I | W | I | F | SEQ ID NO:21 |
| I | F | F | W | I | K | SEQ ID NO:23 |
| L | L | L | V | I | A | SEQ ID NO:13 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A ligand less than about 6 kDa, which binds to a polypeptide comprising the amino acid sequence GWGQPHGG (SEQ ID NO:1) or a polypeptide analog comprising amino acid analogs that are retro-inverso isomers of the amino acid sequence GWGQPHGG (SEQ ID NO:1).

2. The ligand of claim 1, wherein said ligand is less than about 4 kDa.

3. The ligand of claim 1, wherein said ligand is a nucleic acid or nucleic acid analog.

4. The ligand of claim 1, wherein said ligand is a carbohydrate.

5. The ligand of claim 1, wherein said ligand is a peptide or peptidomimetic.

6. The ligand of claim 1, wherein said ligand includes a moiety selected from the group consisting of a porphyrin ring, a phthalocyanine, a napthoquinone, an imidazole, a purine, or a pyrimidine.

7. The ligand of claim 1, wherein said ligand is a peptide ligand less than 20 amino acids in length.

8. The peptide ligand of claim 7, wherein said polypeptide comprises the amino acid sequence GWGQPHGGGWGQPHGG (SEQ ID NO:2).

9. The peptide ligand of claim 7, wherein said polypeptide comprises a retro-inverso isomer of the amino acid sequence D(GGHPQGWG) (SEQ ID NO:39).

10. The peptide ligand of claim 7, wherein the amino acid sequence of said peptide ligand is not present in a streptavidin polypeptide.

11. The peptide ligand of claim 7, wherein said peptide ligand binds to said polypeptide in the presence of a metal.

12. The peptide ligand of claim 11, wherein said metal is copper.

13. The peptide ligand of claim 12, wherein copper is present at a concentration from about 100 nM to about 500 μM.

14. The peptide ligand of claim 12, wherein copper is present at a concentration from about 500 nM to about 200 μM.

15. The peptide ligand of claim 7, wherein said peptide ligand includes the amino acid sequence X₁X₂X₃X₄X₅X₆, provided that
X₁ is L, W, or I;
X₂ is L, Q, F, or L;
X₃ is I, Y, V, F, or L;
X₄ is W or V;
X₅ is I; and
X₆ is P, A, F, K, or A.

16. The peptide ligand of claim 15, wherein said peptide ligand comprises an amino acid sequence selected from the group consisting of LLIWIP (SEQ ID NO:3), WLYWIP (SEQ ID NO:4), WLVWIA (SEQ ID NO:27), IQIWIF (SEQ ID NO:21), IFFWIK (SEQ ID NO:23), and LLLVIA (SEQ ID NO:13).

17. The peptide ligand of claim 7, wherein said peptide ligand comprises the amino acid sequence of a peptide of Table 1 (SEQ IDs 3-30).

18. The peptide ligand of claim 7, wherein said peptide ligand is less than 15 amino acids in length.

19. A polypeptide comprising the amino acid sequence of two or more of the peptide ligands of claim 7.

20. A composition comprising the peptide ligand of claim 7.

21. The composition of claim 20, wherein said composition comprises a solid support.

22. The composition of claim 21, wherein said peptide ligand is coupled to said solid support.

23. The composition of claim 22, wherein said solid support is a resin.

24. The composition of claim 22, wherein said solid support is a membrane.

25. A composition comprising the polypeptide of claim 19.

26. The composition of claim 25, wherein said composition comprises a solid support.

27. The composition of claim 26, wherein said peptide is coupled to said solid support.

28. The composition of claim 27, wherein said solid support is a resin.

29. The composition of claim 27, wherein said solid support is a membrane.

30. A method of identifying a ligand for a prion protein, the method comprising:
a) contacting a test agent with a peptide comprising at least four continuous amino acids of the sequence GWGQPHGGGWGQPHGG (SEQ ID NO:2), or at least four continuos monomers of or a polypeptide analog comprising amino acid analogs that are retro-inverso isomers of the amino acid sequence GWGQPHGG (SEQ ID NO:1); and
b) detecting a complex comprising the test agent and said polypeptide, thereby identifying a ligand for a prion protein.

31. The method of claim 30, wherein the peptide comprises at least five continuous amino acids of the sequence GWGQPHGGGWGQPHGG (SEQ ID NO:2).

32. A method of identifying a ligand for a prion protein, the method comprising:
a) contacting a test agent with a peptide comprising at least three continuous D-amino acids of the sequence D(GGHPQGWGGGHPQGWG) (SEQ ID NO:34); and
b) detecting a complex comprising the test agent and said polypeptide, thereby identifying a ligand for a prion protein.

33. The method of claim 32, wherein the peptide comprises at least four continuous D-amino acids of the sequence D(GGHPQGWGGGHPQGWG) (SEQ ID NO:34).

34. The method of claim 32, wherein said test agent is a selected from the group consisting of a polypeptide, peptide, peptidomimetic, small organic molecule, small inorganic molecule, nucleic acid, lipid, and a carbohydrate.

35. The method of claim 32, wherein said test agent and polypeptide are contacted in the presence of a metal.

36. The method of claim 35, wherein said metal is copper.

37. The method of claim 36, wherein said copper is present in a concentration of 100 nM to about 500 μM.

38. The method of claim 36, wherein said copper is present at a concentration from about 500 nM to about 200 μM.

39. A ligand identified according to the method of claim 32.

40. A method of detecting the presence of a prion protein in a biological fluid, the method comprising
a) contacting the biological fluid with the ligand of claim 1 under conditions sufficient to cause formation of a complex between said prion protein, if present, in said biological fluid and said peptide; and
b) detecting said complex, thereby detecting the presence of a prion protein in said biological fluid.

41. The method of claim 40, wherein said biological fluid is selected from the group consisting of blood, plasma, serum, cerebrospinal fluid, urine, saliva, milk, ductal fluid, tears, and semen.

42. A method of detecting the presence of a prion protein in an environmental sample, the method comprising
a) contacting the environmental sample with the ligand of claim 1 under conditions sufficient to cause formation of a complex between said prion protein, if present, in said sample and said ligand; and
b) detecting said complex, thereby detecting the presence of a prion protein in said sample.

43. The method of claim 42, wherein said environmental sample comprises the water-soluble extract of a solid environmental sample.

44. The method of claim 43 wherein said environmental sample comprises soil, grass or hay.

45. A method of removing a prion from a biological fluid, the method comprising
a) contacting the biological fluid with the ligand of claim 1 under conditions sufficient to cause formation of a complex between said prion, if present, in said biological fluid and said ligand; and
b) removing said complex from said biological fluid, thereby removing said target from said biological fluid.

46. The method of claim 45, wherein said biological fluid is selected from the group consisting of blood, plasma, serum, cerebrospinal fluid, urine, saliva, milk, ductal fluid, tears, and semen.

47. The method of claim 45, wherein said peptide ligand is coupled to a solid support.

48. A method of removing a prion from an environmental sample, the method comprising
a) contacting the sample with the ligand of claim 1 under conditions sufficient to cause formation of a complex between said prion, if present, in said biological fluid and said ligand; and
b) removing said complex from said environmental sample, thereby removing said prion from said environmental sample.

49. The method of claim 48, wherein said environmental sample is selected from the group consisting of soil, hay, the soluble component of soil, and the soluble component of hay.

50. The method of claim 48, wherein said peptide ligand is coupled to a solid support.

51. A method of treating or retarding the development of a prion-associated pathology in a subject, the method comprising administering to said subject the ligand of claim 1 in an amount sufficient to treat or retard the development of said pathology.

52. The method of claim 51, wherein said subject is a human.

53. The method of claim 51, wherein said pathology is Creutzfeldt-Jakob disease, Gerstmann-Straussler-Scheinker disease, fatal familial insomnia, scrapie, bovine spongiform encephalopathy, transmissible mink encephalopathy, feline spongiform encephalopathy, exotic ungulate encephalopathy and chronic wasting disease.

54. The method of claim 53, wherein said pathology is Creutzfeldt-Jakob disease.

55. The method of claim 54, wherein the Creutzfeldt-Jakob disease is iatrogenic, new variant, familial, or sporadic Creutzfeldt-Jakob disease.

56. A method of detecting a prion in a sample, the method comprising:
a) contacting a sample known to or suspected of containing a prion protein with an affinity absorbent comprising the ligand of claim 1 under conditions that allow for the formation of a first complex between said ligand and prion, if present;
b) adding an anti-prion antibody under conditions allowing for formation of a second complex between said first complex and said anti-prion antibody; and
c) detecting said second complex, thereby detecting said prion.

57. The method of claim 56, wherein said sample is a biological fluid.

58. The method of claim 56, wherein said sample is an environmental fluid.

59. The method of claim 56, wherein said sample is soil.

60. The method of claim 56, further comprising concentrating said prion protein in said sample prior to contacting said sample with said absorbent.

61. The method of claim 56, further comprising washing said first complex prior to adding said anti-prion antibody.

62. The method of claim 56, wherein said immunoabsorbent is present on a bead.

63. The method of claim 56, wherein said second complex is detected by chemiluminescence.

64. The method of claim 62, wherein the signal associated with second complex is chemiluminescence.

65. The method of claim 56, wherein the signal associated with said second complex is compared to the level of the signal associated with second complex obtained by incubating said anti-prion antibody in a control solution lacking said sample.

66. The method of claim 56, wherein the signal associated with said second complex is compared to the level of the signal associated with second complex obtained by incubating said sample in a solution lacking said anti-prion antibody.

67. A method of detecting the presence of a prion protein in a biological fluid, the method comprising
a) contacting the biological fluid with an amino resin under conditions sufficient to cause formation of a complex between said prion protein, if present, in said biological fluid and said resin; and
b) detecting said complex, thereby detecting the presence of a prion protein in said biological fluid.

68. The method of claim 67, wherein said biological fluid is selected from the group consisting of blood, plasma, serum, cerebrospinal fluid, urine, saliva, milk, ductal fluid, tears, and semen.

69. A method of detecting the presence of a prion protein in an environmental sample, the method comprising
a) contacting the environmental sample an amino resin under conditions sufficient to cause formation of a complex between said prion protein, if present, in said sample and said amino resin; and
b) detecting said complex, thereby detecting the presence of a prion protein in said sample.

70. The method of claim 69, wherein said environmental sample comprises the water-soluble portion of a solid environmental sample.

71. The method of claim 70 wherein said environmental sample comprises soil, grass or hay.

72. A method of removing a prion from a biological fluid, the method comprising
a) contacting the biological fluid with an amino resin under conditions sufficient to cause formation of a complex between said prion, if present, in said biological fluid and said amino resin and
b) removing said complex from said biological fluid, thereby removing said target from said biological fluid.

73. The method of claim 72, wherein said biological fluid is selected from the group consisting of blood, blood compositions, serum, cerebrospinal fluid, urine, saliva, milk, ductal fluid, tears, and semen.

74. A method of removing a prion from an environmental sample, the method comprising
a) contacting the sample with an amino resin under conditions sufficient to cause formation of a complex between said prion, if present, in said environmental sample and said resin; and
b) removing said complex from said environmental sample, thereby removing said prion from said environmental sample.

75. The method of claim 74, wherein said environmental sample is selected from the group consisting of soil, hay, the soluble component of soil, and the soluble component of hay.


SEQUENCE LISTING (40 sequences)

SEQ ID NO: 1 - protein, 8 aa, Artificial Sequence
  Description of Artificial Sequence Prion Protein Amino Acid Sequence
GWGQPHGG

SEQ ID NO: 2 - protein, 16 aa, Artificial Sequence
  Description of Artificial Sequence Prion Protein Amino Acid Sequence
GWGQPHGGGWGQPHGG

SEQ ID NO: 3 - protein, 6 aa, Artificial Sequence
  Description of Artificial Sequence Synthetic Peptide Sequence
LLIWIP

SEQ ID NO: 4 - protein, 6 aa, Artificial Sequence
  Description of Artificial Sequence  Synthetic Peptide Sequence
WLYWIP

SEQ ID NO: 5 - protein, 6 aa, Artificial Sequence
  Description of Artificial Sequence Synthetic Peptide Sequence
WEFYWF

SEQ ID NO: 6 - protein, 6 aa, Artificial Sequence
  Description of Artificial Sequence Synthetic Peptide Sequence
YVFNWY

SEQ ID NO: 7 - protein, 6 aa, Artificial Sequence
  Description of Artificial Sequence Synthetic Peptide Sequence
LAWFWR

SEQ ID NO: 8 - protein, 6 aa, Artificial Sequence
  Description of Artificial Sequence Synthetic Peptide Sequence
GFFFWW

SEQ ID NO: 9 - protein, 6 aa, Artificial Sequence
  Description of Artificial Sequence Synthetic Peptide Sequence
FYVFTA

SEQ ID NO: 10 - protein, 6 aa, Artificial Sequence
  Description of Artificial Sequence Synthetic Peptide Sequence
YFIWWE

SEQ ID NO: 11 - protein, 6 aa, Artificial Sequence
  Description of Artificial Sequence Synthetic Peptide Sequence
SFPYYY

SEQ ID NO: 12 - protein, 6 aa, Artificial Sequence
  Description of Artificial Sequence Synthetic Peptide Sequence
LEIRLA

SEQ ID NO: 13 - protein, 6 aa, Artificial Sequence
  Description of Artificial Sequence Synthetic Peptide Sequence
LLLVIA

SEQ ID NO: 14 - protein, 6 aa, Artificial Sequence
  Description of Artificial Sequence Synthetic Peptide Sequence
SLEEYV

SEQ ID NO: 15 - protein, 6 aa, Artificial Sequence
  Description of Artificial Sequence Synthetic Peptide Sequence
LRVIIS

SEQ ID NO: 16 - protein, 6 aa, Artificial Sequence
  Description of Artificial Sequence Synthetic Peptide Sequence
QLGHQW

SEQ ID NO: 17 - protein, 6 aa, Artificial Sequence
  Description of Artificial Sequence Synthetic Peptide Sequence
SNYGPY

SEQ ID NO: 18 - protein, 5 aa, Artificial Sequence
  Description of Artificial Sequence Synthetic Peptide Sequence
PFHPG

SEQ ID NO: 19 - protein, 6 aa, Artificial Sequence
  Description of Artificial Sequence Synthetic Peptide Sequence
WIPPYN

SEQ ID NO: 20 - protein, 6 aa, Artificial Sequence
  Description of Artificial Sequence Synthetic Peptide Sequence
WFPHFF

SEQ ID NO: 21 - protein, 6 aa, Artificial Sequence
  Description of Artificial Sequence Synthetic Peptide Sequence
IQIWIF

SEQ ID NO: 22 - protein, 6 aa, Artificial Sequence
  Description of Artificial Sequence Synthetic Peptide Sequence
LWWLFV

SEQ ID NO: 23 - protein, 6 aa, Artificial Sequence
  Description of Artificial Sequence Synthetic Peptide Sequence
IFFWIK

SEQ ID NO: 24 - protein, 6 aa, Artificial Sequence
  Description of Artificial Sequence Synthetic Peptide Sequence
RWIISL

SEQ ID NO: 25 - protein, 6 aa, Artificial Sequence
  Description of Artificial Sequence Synthetic Peptide Sequence
QWWFII

SEQ ID NO: 26 - protein, 6 aa, Artificial Sequence
  Description of Artificial Sequence Synthetic Peptide Sequence
VFEYIK

SEQ ID NO: 27 - protein, 6 aa, Artificial Sequence
  Description of Artificial Sequence Synthetic Peptide Sequence
WLVWIA

SEQ ID NO: 28 - protein, 6 aa, Artificial Sequence
  Description of Artificial Sequence Synthetic Peptide Sequence
YWFIYI

SEQ ID NO: 29 - protein, 6 aa, Artificial Sequence
  Description of Artificial Sequence Synthetic Peptide Sequence
TGIPII

SEQ ID NO: 30 - protein, 6 aa, Artificial Sequence
  Description of Artificial Sequence Synthetic Peptide Sequence
HKEQGA

SEQ ID NO: 31 - protein, 5 aa, Artificial Sequence
  Description of Artificial Sequence Fragment of Prion Protein Amino Acid Sequence
HGGGW

SEQ ID NO: 32 - protein, 4 aa, Artificial Sequence
  Description of Artificial Sequence Prion Protein Copper Binding Amino Acid Motif
HGGG

SEQ ID NO: 33 - protein, 16 aa, Artificial Sequence
  Description of Artificial Sequence Prion Protein Repeated Amino Acid Sequence
PHGGGWGQPHGGGWGQ

SEQ ID NO: 34 - protein, 17 aa, Artificial Sequence
  Description of Artificial Sequence Prion Protein Retro-Inverso Amino Acid Sequence
DGGHPQGWGGGHPQGWG

SEQ ID NO: 35 - protein, 6 aa, Artificial Sequence
  Description of Artificial Sequence Peptide 76
FLLHPI

SEQ ID NO: 36 - protein, 6 aa, Artificial Sequence
  Description of Artificial Sequence Peptide 55
HHHPQT

SEQ ID NO: 37 - protein, 6 aa, Artificial Sequence
  Description of Artificial Sequence Peptide 71
RYHVYF

SEQ ID NO: 38 - protein, 6 aa, Artificial Sequence
  Description of Artificial Sequence Prion Binding Ligand Peptide Retro-Inverso Sequence
FIWIQI

SEQ ID NO: 39 - protein, 4 aa, Artificial Sequence
  Description of Artificial Sequence 3F4 Antibody Recognized Prion Protein Peptide Sequence
MKHM

SEQ ID NO: 40 - protein, 6 aa, Artificial Sequence
  Description of Artificial Sequence Prion Protein Amino Acid Sequence
XXXXXX